(19) United States
(12) Patent Application Publication
(10) Pub. No.: US 2004/0087928 A1
(43) Pub. Date: May 6, 2004

(54) Method of making preformed absorbent cores and absorbent cores produced thereby

(76) Inventor: Paul M. Ducker, St. Simons, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/283,998

(22) Filed: Oct. 30, 2002

(51) Int. Cl. A61F 13/15; A61F 13/20

(52) U.S. Cl. 604/385.01

(57) ABSTRACT

A method of forming preformed absorbent cores wherein the absorbent cores have material properties which vary within the body of the core. The cores are formed by controlling the output of forming heads upon an air-laid non-woven apparatus. The forming heads or other particle distribution units distribute the fibers or particles upon the web such that alternating regions of material properties are created across the web. After each fiber disposition step, the web is optionally densified and the web is slit into several smaller web segments. The web is differentially densified and optionally differentially embossed such that the resulting web has a uniform thickness after being layered into a bundle. The uniform thickness of the web allows the finished non-woven web to be bundled for storage or transportation of the web without problems previously associated with layering webs having non-uniform material profiles.

### FIELD OF THE INVENTION

This invention relates to absorbent cores for use in unitary absorbent articles such as disposable diapers, sanitary napkins, training pants, and the like. The invention further relates to preformed cores for use in such articles.

### BACKGROUND OF THE INVENTION

Absorbent articles have evolved into complex absorbent systems. The absorbent systems are based upon highly absorbent cores formed from non-woven materials. The non-woven core is typically a defiberized paper pulp mat formed by feeding fluff pulp sheets into a defiberizing mill and suspending the individualized fibers into an airstream. In traditional absorbent article production, the airstream is directed at a moving forming wire or a forming wire with a layer of porous tissue on top. A vacuum below the wire extracts the air causing the fibers to form a continuously moving fluff mat. The mat is then cut into individual cores and combined with the rest of the product components later in a converting process. Construction of the unitary article takes place in a series of closely related process steps from raw materials to the finished product.

It is often desired to vary properties of the absorbent core across the area or through the thickness of the core. For instance, the use of core mats having a nonuniform composition makes the cores easier to fold. Folding an absorbent core in low basis weight zones is much easier than running folds through high basis weight portions of the core. Second, there are zones of a wearer's body which are dynamic and zones which are static as taught by U.S. Pat. No. 5,669,897 to Lavon et al. Placing flexible core material in dynamic regions and less flexible core materials in the static regions allows the core to be most comfortable and follow the motions of the body most closely.

Additionally, the absorbency characteristics of the core may be varied by gradating fiber basis weight, fiber thickness, density of the core, addition of different types of fibers, and/or the addition of superabsorbent polymers (SAP). Additives or treated fibers may also be preferentially dispersed within areas of the core. Different types of fibers include chemically modified cellulosic fibers which may be dispersed within the target zone of the product. Modified cellulosic fibers include cross-linked stiffened twisted cellulosic fiber as taught by U.S. Pat. No. 5,998,511.

Another method of web production which allows a specific targeting of core properties is the use of a drum former. Instead of forming a continuous fluff mat on a moving wire, a drum former is a large vacuum drum with 3-dimensional molds called pockets arrayed around it's circumference. These pockets have screens which are formed to the 3-dimensional shape desired, for the laydown of fluff and SAP for a given core design. Fluff from the defiberizing mill are transported by vacuum and deposited in the pockets of the rotating forming drum. A high speed pin roll then removes the excess fluff from the top of the pockets making the upper surface of the fluff in the pocket flat. A reversal of vacuum in one portion of the drum rotation causes the fluff mat in the pockets to separate from the drum and be transported down a belt or screen as a continuous web. The web can have an anatomically shaped dimensional outline and a basis weight profile on both the cross machine direction and in the machine direction. In some applications, the cores leave the drum connected together as a continuous web and in other applications, the cores are removed from the drum as discrete cores requiring no other cutting operations.

Recently, the absorbent products industry has moved to the use of preformed absorbent cores in the construction of absorbent articles rather than constructing the absorbent articles as unitary articles from raw materials. This move is due to several factors. First, converting machines involve a multitude of unit operations which increase as the complexity of absorbent products increases. This increase in serial unit operations makes it more likely that the machine will have to be taken offline due to process or material problems. Supplying preformed core material to the conversion equipment simplifies the converting process and makes delays due to equipment failure less likely.

Second, absorbent cores continue to move towards thinner and more flexible designs utilizing more gram-efficient materials. Processes that are specially designed for producing thinner materials are not necessarily compatible with serial converting processes. For example, latex application and through-air bonding steps in high density core production are not suitable to processes which are often halted and restarted, such as conversion machines which must be shut down often.

Third, preformed cores may be produced on wide web machines since they do not have incompatible processes, such as fastener application, that the final converting process has. After production of the wide web, the web may be slit into many individual webs. This configuration allows a single wide-web machine to provide material fast enough to supply many converting machines and do so in a more efficient manner.

Finally, preformed cores can be of relatively high density and this has a positive impact on shipping cost. With high density preformed cores, the core material and the finished cores are of similar density, in the range of 0.25 g/cm³ to 0.50 g/cm³, and the high density cores are very attractive from the standpoint of shipping and storage relative to preformed cores of lower density. Since the customer, typically a retailer, has a great deal to say about the value of a product and is interested in reducing its storage costs and shelf space requirements, it is even more advantageous to have a high density core in a finished absorbent article.

Though the use of preformed cores has become more popular, absorbent articles using preformed cores currently do not display the high degree of evolution which those cores produced in-line with conversion equipment had previously achieved. For instance, single-piece preformed cores having gradients in shape, basis weight, density, and material composition are not currently available as pre-formed cores. It would be highly desirable to have a preformed absorbent core having gradients of material properties formed therein which could conveniently be stored and transported to an absorbent article manufacturer for use in the conversion of unitary absorbent articles.

To the extent that preformed absorbent cores having gradients of material properties are available, the cores often have different thicknesses or other physical characteristics which make them unsuitable for collecting in layered bundles such as rolls, spools, or festoons. When the uneven non-uniform webs are wound upon rolls or spools, or are layered into festoons, the differential magnitude of the uneven portions of the web tend to multiply, thus creating undulating, uneven, or otherwise misshaped bundles which are unwieldy during handling and unstable during transportation. Such layered bundles are unsuitable for use by absorbent article manufacturers.

Another benefit of preformed cores is the ability to generate large count stacks of absorbent articles in a package, due to the inherent thinness of the preformed cores. However, any differences in core thickness across the face of the folded core are multiplied as the articles are stacked. This difference in core thickness across the face of the core can cause tilted, football-shaped, or unstable stacks of material which negatively affect the appearance of the package as well as the efficiency of the stacking and packaging operation.

What is needed is the ability to produce a preformed core which overcomes the limitations previously encountered with the use of preformed cores in conversion and in storage and transportation. What is further needed is a preformed core which provides all of the advantages of preformed cores, such as the ability to remove core production steps from the conversion process and the ability to make and store large numbers of cores before they are needed in the conversion process, without the limitations of previous preformed cores.

### SUMMARY OF THE INVENTION

One aspect of the present invention is a method of producing high density fibrous preformed absorbent cores having a non-uniform basis weight, density, or composition but advantageously having a uniform caliper (thickness). The preformed cores have different material properties at different locations in the core, but are selectively densified to cause the core to have a uniform caliper. If differential densification alone is unsatisfactory, the web may be differentially embossed to cause the core to have a uniform apparent caliper. The preformed cores are suitable for a number of uses including use as the absorbent component in unitary absorbent articles, such as disposable diapers, sanitary napkins, adult incontinent products, disposable training pants, and the like.

To produce the cores, a web of non-woven material is produced on a wide-web machine. Operating conditions of the wide web machine are varied such that properties of the non-woven web vary in the cross-machine direction. The variation in web properties may be obtained by blocking the output of forming heads in certain regions of the sheet, adjusting the output of forming heads, blocking sections of vacuum behind the forming wire, or any other manner of adjusting the formation of the web.

A wide web can be produced such that material properties of the web vary in a regular pattern over the cross-machine direction of the web. The web is then slit into a number of web segments such that each of the web segments has a variation in material properties in the cross direction, and a plurality of web segments having similar variations in material properties are produced from a single wide web.

To provide better storage and transportation of the web segments, the web segments are processed so that each of the segments is of a uniform caliper, either as a free-standing web or when layered into a bundle of material such as by winding into a roll, spooling, or festooning, despite the variation of basis weight, density, or other properties within the segment. By differentially densifying the web so that the web has a uniform caliper prior to layering, or so that the web attains a uniform caliper when subjected to the pressure of layering, the web segments may be layered such that a stable and uniform layered bundle is formed. Processing of the web segments into a uniform caliper prevents formation of uneven and unstable bundles which would be problematic of layered bundles having non-uniform caliper.

Manipulating the web segment into a uniform caliper may occur with two different types of process operations, differential densification and differential embossing. Differential densification occurs by densifying the non-woven material accross the the web segment such that the resulting web segment has a uniform caliper. This is accomplished by differentially applying pressure across the web segment, such as by a calender roll having differential diameter, or by the differential application of heat or chemicals which cause the web segment to compact into a web of uniform caliper.

In some cases, a web segment that has been densified to a uniform caliper has low basis weight sections with very low density. These low density sections tend to be weak and may not wick liquid very well. In such cases, the low basis weight regions are densified to a caliper corresponding to a basis weight or density which provides appropriate strength and wicking ability. These regions are then embossed to provide an apparent caliper equal to the uniform caliper of the web segment. Thus, the web segment has the advantage of having zones of differing basis weight, density, or material properties across the web, but has a uniform caliper or uniform apparent caliper to provide for easy manipulation, storage, and transportation of the preformed cores. The web may be embossed such that the web does not have a uniform apparent caliper prior to layering, but such that the web attains a uniform apparent caliper after layering into a bundle.

The web segments having varying material properties may be cross-cut to form unitary preformed cores for use in absorbent article production. Use of the preformed core allows production of the core offsite and storage of the core material at the conversion location. Since the conversion equipment does not have to produce the core from scratch, the number of converting steps is reduced and the conversion operation becomes considerably more reliable and, therefore, more cost efficient. Additional improvements in efficiency are obtained due to the uniform apparent caliper of the core materials and the improved stackability of the finished articles.

### DETAILED DESCRIPTION OF THE DRAWINGS

Having thus described the invention in general terms, reference will now be made to the accompanying drawings, which are not necessarily drawn to scale, and wherein:

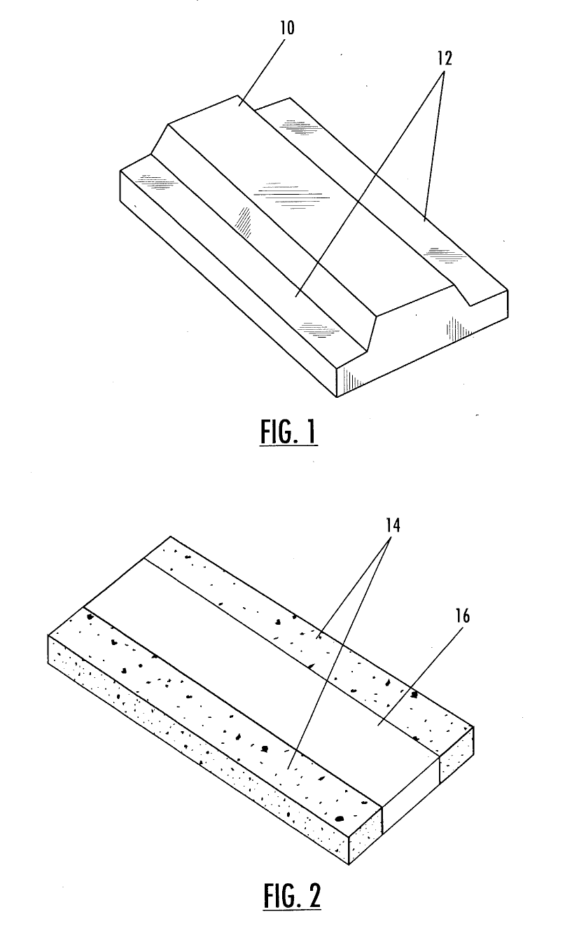

FIG. 1 is a view of the preformed core in accordance with an embodiment of the invention, prior to differential densification,

FIG. 2 is a view of the preformed core in accordance with a second embodiment of the invention,

FIG. 3 is a view of the preformed core in accordance with a third embodiment of the invention,

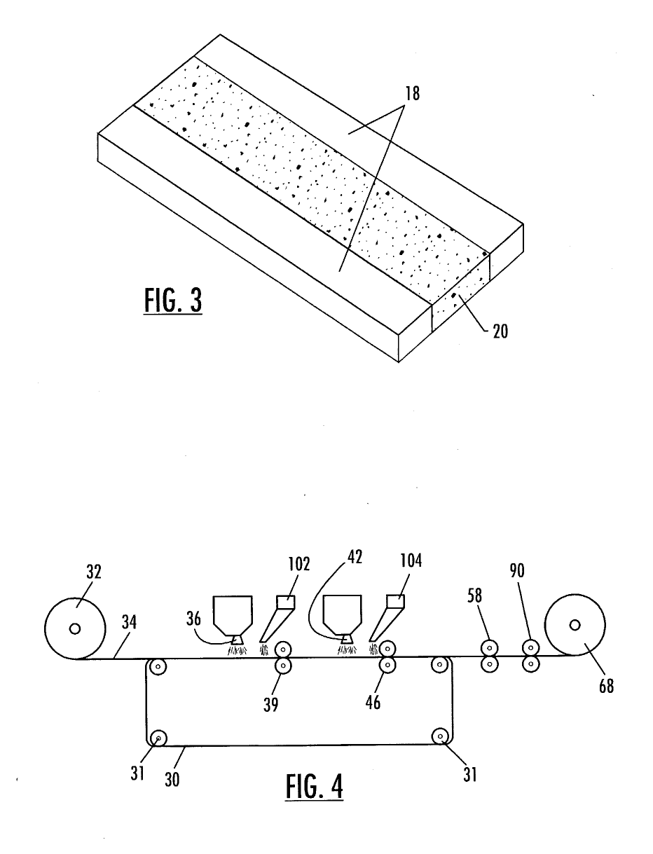

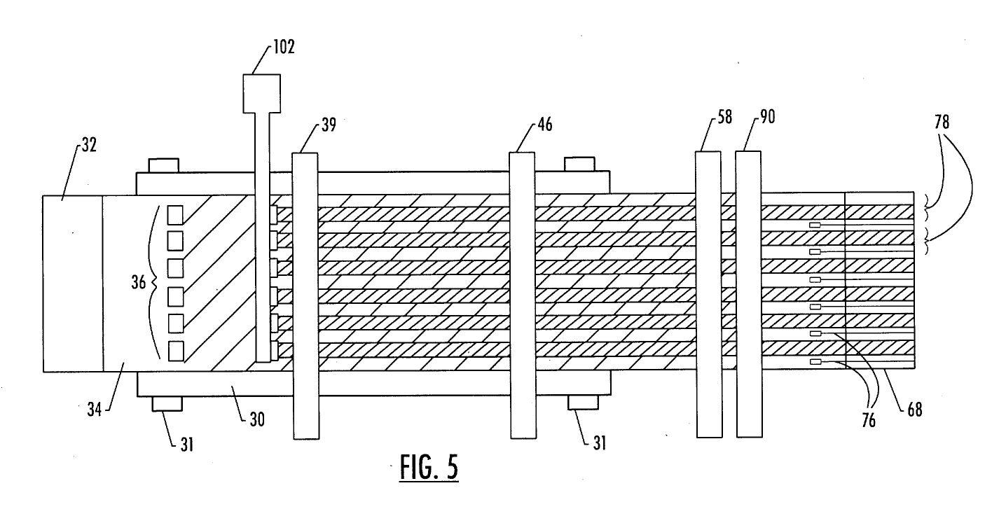

FIG. 4 is a side schematic view of an apparatus for constructing a preformed core in accordance with an embodiment of the invented method,

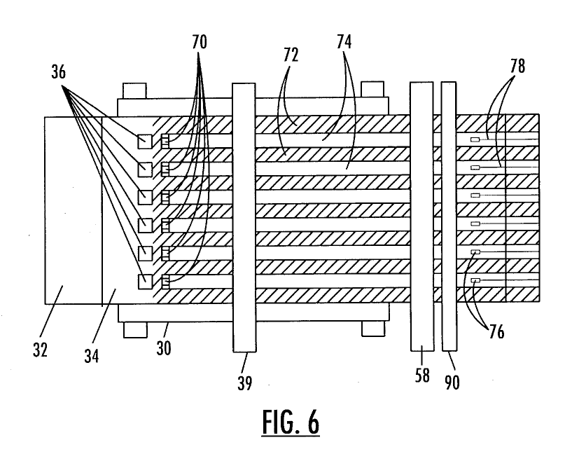

FIG. 5 is a top schematic view of an apparatus for constructing a preformed core in accordance with an embodiment of the invented method,

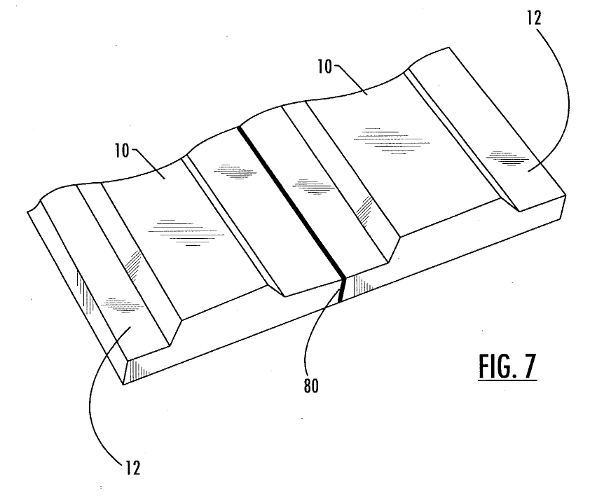

FIG. 6 is a top schematic view of an apparatus for constructing a preformed core in accordance with another embodiment of the invented method,

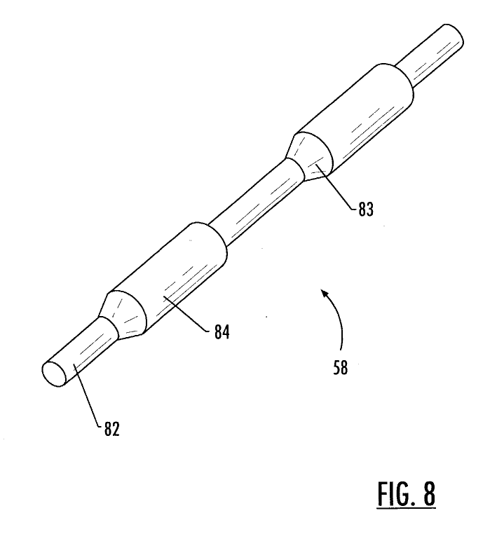

FIG. 7 is a view of webs produced by an embodiment of the invented method prior to differential densification,

FIG. 8 is a view of a differential densification calender roll for use in the invented method,

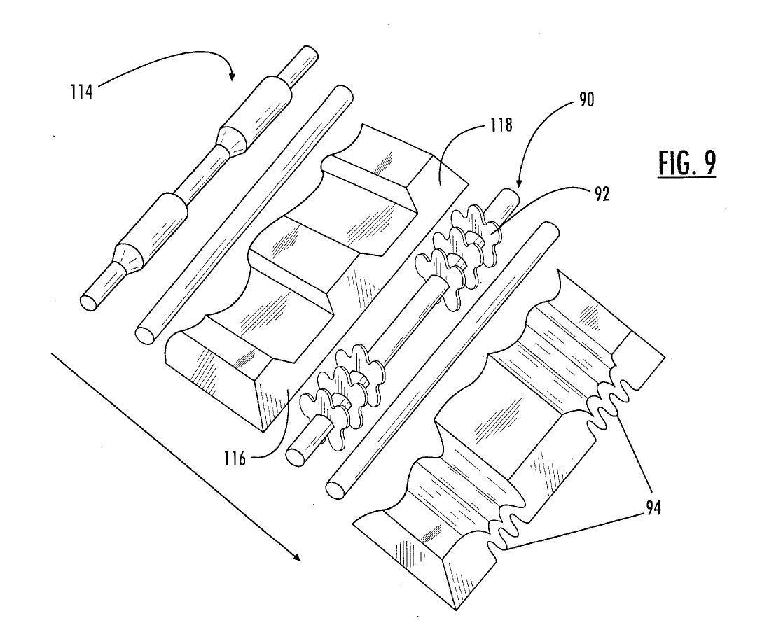

FIG. 9 is a representation of a differential densification calender roll in conjunction with a differential embossing roll in accordance with one embodiment of the invention,

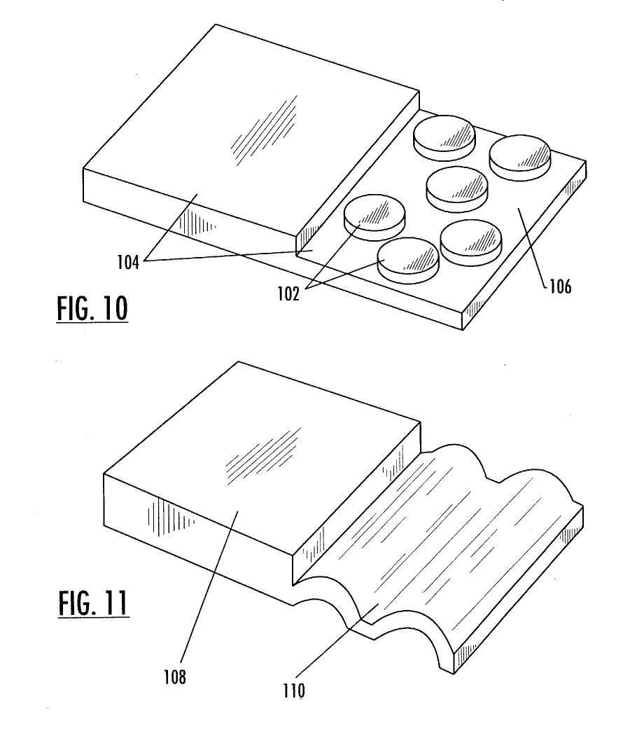

FIG. 10 is a representation of an embodiment of the invention that has been differentially densified using a patterned densification in order to allow for a higher caliper and a higher density in at least some portions of the lower density zone,

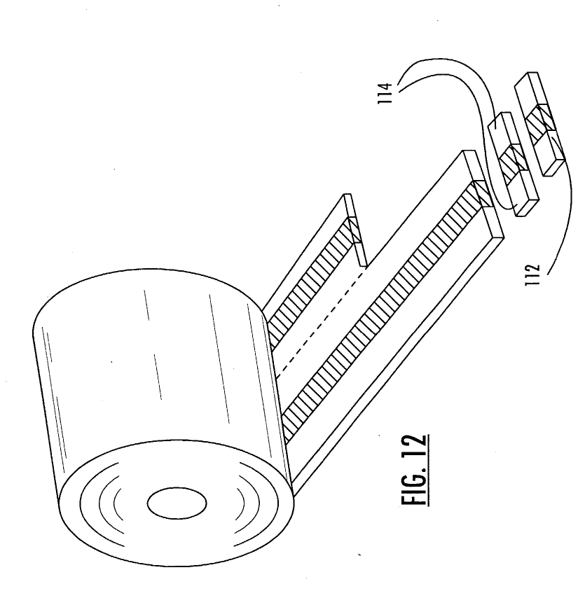

FIG. 11 is a representation of an embodiment of the invention where an absorbent material has been differentially densified to yield a uniform true material density and then the apparent caliper was increased to yield a uniform apparent caliper across the face of the sheet by corrugating the sheet, and

FIG. 12 is a representation of a wide-web roll good with a high basis weight zone in the targeted area suitable for use in a transverse core training pant application.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Referring to FIGS. 1-3, one aspect of the present invention is directed to preformed absorbent cores, generally having a high density, for use in absorbent products with material properties which differ through at least one dimension of the core body. The preformed core has gradations in basis weight, density, and/or composition. The gradations may be in the transverse or longitudinal direction, depending upon the orientation of the core with respect to the absorbent article which incorporates the core.

Since core material located in different positions within absorbent products have different functional requirements, it is advantageous to provide a preformed core material that is already differentiated according to these requirements. While the preformed cores are particularly well suited to application in personal care absorbent products such as diapers and sanitary napkins, the cores may be used in a wide range of absorbent article applications.

Referring again to FIG. 1, an embodiment of the preformed core, shown prior to any differential densification or embossing steps, has a gradation in basis weight. The core has at least one region having a first basis weight in contact with at least one region having a second basis weight lower than the first basis weight. In a preferred embodiment, the core has at least one central region 10 of high basis weight bordered on both lateral edges by regions 12 of low basis weight. For instance, in diaper applications, the high basis weight zone may be as high as 1500 gsm (grams per square meter) or even higher while the low basis weight zones may be as low as 100 gsm. More typically, basis weight range for a diaper is from about 200 gsm to about 1000 gsm. In sanitary napkin applications, high basis weight zones may be 400 gsm and low basis weight zones may be around 60 gsm.

Referring again to FIG. 2, an embodiment of the preformed core has a gradation of density. The core has at least one region having a first density in contact with at least one region having a second density greater than the first density. In a preferred embodiment, the core has at least one central region 16 of a first density and two lateral regions 14 of a second density lower than the first density. Density for the central region for such a core may be as high as 0.50 g/cm³ although for many conventional core materials, the core becomes unacceptably stiff at densities much higher than that. The lower density lateral regions which must be more flexible may have a density as low as 0.04 g/cm³ although regions of very low density, below about 0.10 g/cm³, are weak and must be stabilized prior to further processing. Typical densities vary widely, depending on the materials used and the design requirements for the particular product. Articles, as shown in FIG. 2, produced with a uniform caliper in accordance with the invention have a basis weight which is proportional to the density throughout the article.

Referring again to FIG. 3, an embodiment of the preformed core has a gradation of composition. The core has at least one region having a first composition in contact with at least one region having a second composition different than the first composition. In a preferred embodiment, the core has at least two lateral regions 18 of a particular composition and a central region 20 having especially absorbent materials incorporated therein. Thus, the central region may be designed to incorporate materials with better absorption properties, such as super-absorbent polymers, while the lateral regions may incorporate materials with greater flexibility, such as chemically treated cellulosic fibers, given that the lateral zones frequently must deform or flex with the motion of the wearer. Alternatively, the central zone may incorporate materials that have a better acquisition rate, such as polyester fibers and the lateral edges contain materials that are better at fluid transport, such as fluff pulp.

The material profile of the preformed cores may contain gradation of any or a combination of basis weight, density, composition, and any other material property or structure which may be important to the performance of the core. The specific profile of the preformed core is dictated by the desired end use of the absorbent article to be used with the core.

In both urine and feminine hygiene applications, there is a target zone where liquid typically enters the absorbent core. In this target zone, it is most advantageous to include materials which contain a higher degree of permeability and a resilience to wet collapse in order to facilitate the rapid uptake of liquid. This is typically accomplished by using structures made from stiffer, less wettable fibers and superabsorbent polymers that have a higher gel strength and/or a larger particle size. These types of materials are most advantageously disposed on the liner side face of the article in the target insult zone although in some designs they extend entirely through the core or even are disposed to the back of the core.

A second functional requirement of the core is that the distribution of absorbent materials should generally reflect the distribution of the maximum quantity of liquid that each location in the core may encounter in the worst case usage conditions. In most cases, this means that in regions near the target acquisition area, the basis weight of absorbent storage material should most advantageously be higher than it is in peripheral regions of the product where liquid reaches in lesser quantities and only in certain wearer positions or extreme liquid loadings. Again, this basis weight should be distributed in a manner such that the liquid distribution in all expected loading conditions is met by a sufficient distribution of storage material, typically meaning that the central region of the core should have a higher basis weight than the remainder of the core.

A third functional requirement of the core is that certain regions of the core encounter very dynamic body motions and other regions of the core do not encounter a great deal of flexure. This concept is taught by U.S. Pat. No. 5,669,897 to Lavon et al. In the crotch region of a diaper, for example, the center 60 mm to 75 mm about the centerline of the product is a static zone encountering little flexure. Regions outboard of this zone along the lateral edges of the core are probably the most dynamic zones in the diaper which need to move in and out with the motion of the legs without fracturing or applying uncomfortable pressure against the thighs. Similarly, in feminine hygiene products a static zone along the centerline exists and a dynamic zone along the lateral edges again needs to be accommodated in order to provide a product with the greatest comfort. This crotch zone also happens to be in a zone requiring the greatest storage capacity and greatest acquisition rate which tend to be difficult to achieve while retaining dynamics. Therefore maximizing these properties in the static center zone may be very important in a particular design to offset the compromise of these properties in the dynamic lateral edges that must move comfortably with the wearer's legs. To this end, a core having lateral portions that are low basis weight and low or moderate density may be desired.

When comparing the material properties across the body of a preformed core, it is convenient to speak in terms of an “aspect ratio”. For any given property, the aspect ratio of the material is the measured value for the property in a zone of the core where the value is at its maximum divided by the measured value for the property in a zone of the core where the value is at it's minimum. As a convention, aspect ratios are reported as numbers with values of 1.0 or greater. Use of an aspect ratio provides a relatively easy way to measure the degree of targeting for any particular property of interest.

Since basis weight for diapers may vary from 100 gsm to 1500 gsm, it is possible to have basis weight aspect ratios as high as 15. However, in commercial use it is much more practical to have basis weight aspect ratios between about 1 and about 2. Density aspect ratios are also most typically between about 1 and about 2.

The change from a minimum property value to a maximum property value within the core will generally occur over a gradient. Due to the nature of non-woven production, even step changes in production will typically result in non-woven material having at least some gradient properties. It may be useful to describe the core in terms of property gradients and how quickly those gradients take place. The gradient may be expressed as a unit change in a measure of a property per unit of distance within the core. Another way to express the gradient is to measure the aspect ratio and divide that by the distance across which the aspect ratio is achieved. As an example, the basis weight of a non-woven may change anywhere between 1 and 50 grams per square meter (gsm) per millimeter of distance within that web. Typical basis weight gradient for non-wovens are 1 gsm/mm to about 37 gsm/mm.

The rate of change in basis weight or density from one region of the web to another region of the web may be measured in terms of a basis weight aspect ratio gradient or a density aspect ratio gradient. In the manufacture of the web, it is desirable that the method of manufacture have the ability to create high values for the basis weight aspect ratio gradient or a density aspect ratio gradient, meaning that the properties of the web may be changed rapidly over a small distance. In practice, a favorable basis weight aspect ratio gradient for a feminine hygiene product may be as steep as 0.5/mm, though basis weight aspect ratio gradients as low as 0.01/mm may be useful in some absorbent core applications. Similar density aspect ratio gradients may be required. In diapers and training pants, the basis weight aspect ratio gradients required are somewhat less, such as 0.25/mm. The desired density aspect ratio for diapers is about the same as the desired basis weight aspect ratio. Obviously, the required gradient varies with the design of the desired article.

The preformed core is preferably made from a continuous wide-web process. While not being bound to any particular process, an exemplary method of forming such a material is to physically block part of the forming process from occurring in a particular region of the width of the continuous web as it is formed. Other portions of the process can be likewise adjusted in intensity in the cross direction. An example of this would be a gradient in a thermal bonding temperature or a pressure gradient in the cross direction applied by a densification device such as a calender. The material so made is done in a wide-web format and then slit into multiple web segments, each containing the necessary gradients for a particular design. The web segments are preferably identical but may be of different designs, depending on the need to utilize the entire web.

Referring to FIGS. 4 and 5, an embodiment of a wide web apparatus for forming cores in accordance with the invention is shown. A continuous forming wire 30 carries the non-woven material throughout most of the forming process. The forming wire is a flexible mesh screen supported by a number of rollers 31 upon which non-woven fibers are deposited and supported while air is allowed to pass through. If needed, a carrier tissue 34 may be deposited on the forming wire 30 prior to formation of the web to provide physical support for the non-woven material.

A series of forming heads 36 distribute short lengths of non-woven fibers or combinations of different non-woven fibers onto the forming wire 30, thereby forming the non-woven web. The preferred fiber of the invention is a cellulose pulp fiber, though polyester or other polymer fibers may be used in addition to or instead of cellulose pulp fiber. Additives to the web of the airlaid web may also be placed within the web at this point. Additives include but are not limited to superabsorbent polymers (SAP) or chemically modified cellulose fibers.

A plurality of forming heads, typically 6 to 10, distribute the desired fiber onto the forming wire 30. Optionally, one or more sets of additional forming heads 42 are spaced down the web in the machine direction such that the additional forming heads supply fiber for additional layers of the web. The laydown formed by each forming head can be blocked in some zones across the width of the sheet. Preventing formation in portions of the cross machine direction by a given forming head can be achieved by masking the laydown from above the screen, blocking the vacuum under portions of the screen from below, or having portions of the screen itself blocked off. In an alternative configuration, independent forming heads may be arrayed across the width of the web or segmented across the width of the web and the output from those heads may be controlled independently, thus controlling the formation across the width of the web.

Alternatively, formation may be uniform as material is supplied by a unitary forming head which spans the width of the web. Material distributed by the unitary forming head may be blocked by masking the laydown from above the screen, blocking the vacuum under portions of the screen from below, or having portions of the screen itself blocked off. Alternatively, regions of material may be removed from the web after being deposited by the forming head(s) by using high speed pin rolls or other means to remove material as desired. The removed materials can be taken away using vacuum and then recycled into the process allowing it to be re-deposited.

SAP particles or other specialized absorbent materials may optionally be distributed across the web from distribution ports 102, 104 separate from the forming heads. The distribution ports 102,104 may be arranged such that the specialized absorbent materials are distributed in gradients across the machine direction of the web.

After each fiber disposition step, the web is optionally densified. Densification occurs by subjecting the web or portions of the web to pressure and optionally heat. When cellulose forms the major portion of the web, the cellulose fibers within the web resemble short twisted ribbons. When the cellulose fibers are subjected to heat and pressure, the fibers bind to one another and the web is densified. The amount of heat and degree of pressure determine the extent to which the fibers bind to one another and the extent of densification of the web. Pressure is preferably supplied by traveling the web through one or more sets of calender rolls 39. Using the calender rolls 39, the web may be easily compacted to the desired caliper and density.

As previously mentioned, composition of the web may be varied by depositing additional fibers upon the web via additional sets of forming heads 42, and subjecting the web to additional heat and pressure densification steps 46. By using additional sets of forming heads, the material properties of the web may be varied through the caliper of the web. As many sets of forming heads as necessary, each with an appropriate blocking mechanism as previously described, and as many densification devises as needed may be supplied to construct the desired web.

After addition of fiber to the web is complete and the web has been densified to an extent to which the web may support itself without the need of the forming wire 30, the web is transferred from the forming wire. The final web preferably has a density that does not exceed 0.50 g/cm³, to avoid unnecessary stiffness for most conventional materials, although in some cases it is possible to be even higher in density and still be acceptable. Most core designs do not require basis weight aspect ratio gradients higher than 0.5/mm.

During production of the web, the web is heated, preferably by one or more heated calender rolls. Heat may also be supplied by running the web through ovens or any other heating apparatus capable of heating the web. The web is preferably heated to temperatures from about 125° C. to 180° C. for cellulose based webs. Pressure upon the web is typically supplied by running the web through one or more calenders at a pressure of about 0.1 to 10 psi. In order to improve the strength of the sheet, binder fibers may be added. Also, binder is optionally applied to the web during formation of the web, and may be applied through the forming heads or as a separate spray.

The web is preferably slit by slitters 76 into several smaller web segments 78. The slitters 76 are positioned such that each of the smaller web segments 78 has the same material profile, given that certain portions of the web may have to be wasted, depending on the size of the web machine and layout of the desired material profile. In this manner, several identical webs having material properties which vary across the web may be simultaneously created on a single wide-web machine. The finished non-woven web is then wound onto a roll 60 for storage or transportation of the web. The roll of webs may be easily processed as absorbent cores for use by an absorbent article manufacturer.

FIG. 6 shows an exemplary process of blocking the forming heads such that regions of alternating basis weight are formed in the cross-machine direction of the moving web. A number of blocking units 70 are positioned such that they vary the flow of fiber from the forming heads 36 to the underlying web which rests upon the forming wire 30. In this exemplary embodiment, alternating regions of high basis weight 72 and low basis weight 74 are deposited upon the web. The web is further processed as described above through the final calendering step.

It is recognized that collection of material having various basis weights, densities, or compositions upon the web may cause problems when the collection of material involves satisfactorily layering the wide web, or multiple web segments made from the wide web, into a bundle such as by winding onto a roll or spool, or by festooning. Bundling of non-woven material, such as by winding or festooning, is often referred to as “packaging” in the art of non-woven manufacture.

For example, when forming a roll or spool, the various properties of the web and corresponding variation in web caliper under the applied wind tension cause the roll to be uneven and unstable. The resulting rolls and spools may disadvantageously be oval or egg-shaped during the winding process. The varying surface speeds resulting can cause shearing and wrinkling of the material in gradient areas and cause the sheet to track, creating a telescoped roll. Additionally, such poorly formed rolls do not lend themselves well to transportation, storage, and handling. When unwinding, such rolls and spools also cause difficulties with web tracking and that, combined with unsightly wrinkles formed into the rolls negatively affects the quality of the finished articles.

When festooning, which is the process of stacking a continuous web in layers where each layer is folded back on the one below it in a reversing zig-zag pattern, relatively tall columns of layers are formed before the festoon traverses to the next position forming a new column adjacent to the first.

In such situation, or in a relatively wide web festoon, where only a single column is formed, non-uniform web thickness causes the festooned bundle to be very unstable. In festooning schemes where the festoon continually traverses back and forth and columns are not formed, control of the positioning of the web during festooning is often difficult due to the non-uniform thickness of the web.

To solve the problems associated with layered bundle formation, one or more differential densification steps may be used to manipulate the web or webs so that the web has a relatively uniform caliper prior to layering or so that the web has a caliper profile prior to layering which will become uniform when subjected to the pressure exerting on the web by the weight or winding tension of the adjacent layers. By “uniform caliper” it is meant that variation of the web caliper, when layered, is small enough to provide for a substantially uniform layered bundle when the material of the web is layered upon itself. Due to the difference in properties of each region upon the web, each region may have to be subjected to densification independent of other dissimilar regions in order to obtain a uniform overall web caliper. To this end, differential densification treatments are applied to the web at different locations across the web.

Of course, methods other than rolling, spooling, and festooning may be used to layer the continuous web into a bundle suitable for storage and transportation. The differential densification of the invention provides improved layering characteristics for any such layering technique.

There are a significant number of physical effects such as heat, pressure, moisture, texturing, addition of substances, various fields, etc which can have an effect on the caliper of the web. The differential application of any one of these physical effects may be used to achieve a differential densification as required to achieve a sufficiently uniform caliper.

Uniform web caliper may be achieved by use of a calender having rings of differing diameters which correspond to the properties of the web regions being calendered. For instance, FIG. 7 shows a portion of two webs having high basis weight regions 10 and low basis weight regions 12 and separated by a slit 80. An exemplary calender roll 58 is shown in FIG. 8 having small diameter regions 82 and large diameter regions 84 which correspond to the low basis weight regions 12 and high basis weight regions 10 of the web, respectively. Gradients from high 10 to low 12 basis weight regions correspond to tapered regions 83 of the calender roll 58.

The design of the calender 58 will depend upon the properties of the particular web being calendered. The calender is primarily used to compact those regions of the webs having comparatively high basis weight regions. However, it is noted that densification is influenced by the amount of interfiber bonding which occurs in the web during densification. Depending upon the bonding characteristics of each region of the web, those particular regions exhibit positive, negative, or neutral rebound characteristics after being calendered. In other words, the varying regions of the webs will rebound differently in response to being calendered. Therefore, the rebound characteristics of the webs must be considered in order to appropriately densify the webs into uniform caliper.

In typical use, the calender 58 differentially compresses and densifies the web having varying basis weights, densities, and compositions into a web having a uniform caliper. The calendering takes into account any rebounding of the material which may occur and the variation in rebounding associated with different regions of the web.

Calender rolls 58, 90 are not the only means which may be used to differentially densify or to emboss the web. Other methods of applying pressure to the sheet may include moving belts, vacuum and other apparatus. Densification may also be achieved by adding moisture or latex to the cellulosic web, which causes the fibers to lose their rigidity and collapse. Densification can also be accomplished by adding a high density material such as SAP granules in higher concentrations.

In some circumstances, those areas of a uniformly thickened web or web segment having a very low basis weight or very low density are too weak to properly support the web. In other circumstances, areas of very low basis weight or density have very little wicking ability. In such circumstances, the low density or low basis weight portion of the web may be further densified to provide added strength to the web. Thus compacted, the further densified area of the web has a caliper less than that of the otherwise uniform caliper web. After appropriate densification, the now densified low caliper area of the web is preferably subjected to an embossing step where a raised pattern is embossed into the low caliper area. The low caliper area is embossed such that the ridges, crests, or other raised portions of the pattern embossed therein give the embossed region an apparent caliper equal to the otherwise overall caliper of the web. As used herein, “apparent caliper” is the measured distance from the outer most series of ridges, crests, or analogous portions of an embossed pattern on one side of an embossed article to the corresponding outer most series of ridges, crests, or analogous portions of the embossed pattern on the opposing side of that embossed article.

The web produced by the invented method has a substantially uniform thickness when wound on a roll. As used herein “uniform thickness” refers to a web having substantially uniform thickness either as a result of a uniform actual caliper or a uniform apparent caliper resulting from an embossing step. Thickness of the web should be as uniform as possible, but differences in thickness of 20% or less are typically acceptable for bundle formation. Thus, it is favorable if the free-standing web has a uniform thickness as defined herein, but more important that the web has a uniform thickness when subjected to the pressures resulting from a layering operation. The overall goal of differential densification is to control the thickness of the web within a bundle resulting from a layering-type of bundle formation.

Differential densification is preferably accomplished by using a ring-style calender system that involves the use of more than one calender. A conventional calender is used first to densify the web to a degree were the lowest density zones in the web have reached their final design densities. Then a second calender 58 consisting of a shaft with moveable ring segments, mating with a conventional opposite roll, is used to further densify portions of the web that need additional densification. Use of a differential densification calender roll having moveable rings allows the calender to be easily adjusted between runs or between production of different types of webs. Since the rings can be configured to be moveable on the shaft or even removable, it is possible to run a full range of possible web designs. Additionally, using multiple calenders minimizes any issues associated with the mismatch in surface speed between the larger and smaller diameter portions of a single calender.

A portion of the web may be embossed by any industrially useful method of imparting texture to the web. A preferred method of embossing the web is by providing an additional calender, or the same calender 58 as that which provides densification to the web, further having a textured surface. FIG. 9 shows an example of calenders 90 having texture for providing embossing to portions of the web 94 which are of lower caliper than the otherwise uniform caliper of the web. As shown, a differential densification calender roll 114 has been used to densify low basis weight regions 116 of the web to a caliper which is less than the uniform caliper regions 118 of the web. The web is then fed through differential embossing calender rolls 90 having textured surfaces 92 which correspond to the densified low caliper regions 116 of the web. The embossing provides the low caliper portions of the web with an increased apparent caliper. The apparent caliper of the embossed regions 94 of the web approximates the uniform caliper of the unembossed regions of the web such that the web has an overall uniform caliper/apparent caliper.

Referring to FIG. 10, an embodiment of the invention is shown which has embossed surfaces 102 on at least one face of the sheet in order to increase the apparent caliper of the web segment. The web segment consists of a high basis weight region 104 and a low basis weight region 106. Basis weights are uniform within each region. Embossed zones 102 have a lower density, increasing the apparent caliper of the low basis weight zone to the caliper of the high basis weight zone.

Referring to FIG. 11, an exemplary web has a high basis weight region 108, and a low basis weight region 110. Both regions have the same density. The low basis weight region 110 is corrugated in order to make the apparent caliper of the low basis region 110 the same as that of the high basis weight region 108.

Referring to FIG. 12, one aspect of the present invention is directed to preformed cores to be used on transverse core converting machines. This configuration is frequently used to make training pants and sometimes is used to make diaper products as well. The cores are oriented in the transverse direction with regard to the axis of the preformed sheet. These cores travel down the converting machine in a sideways manner so that the transverse direction of the preformed core process becomes the longitudinal direction of the core. FIG. 12 shows a wide-web roll that can be slit into two preformed core rolls. These have a zone corresponding to the target area 112 of a training pant article which has an increased basis weight, relative to the rest 114 of the core. The cores are cut off of the roll in a transverse manner as shown to form the individual cores. One difficulty in executing this design using the prior art is that no means is provided to independently control the caliper of the sheet. Therefore, the high basis weight zone 112 of the sheet would have a higher (or possibly lower, depending on certain process conditions) caliper than the rest of the web. When this is formed into a relatively wide roll, the roll loses its cylindrical shape after just a few winds. Wrinkles form near the transition from high to low basis weight as the differing roll circumferences that form result in a different surface speed across the web causing a shearing to take place in the sheet as it winds. An even greater problem is the fact that the high basis weight zone is offset from the centerline of the sheet. This causes the material to track as it winds, resulting in a telescoped roll. Differential densification and, optionally, differential embossing, as described in detail above, overcome the problems previously associated with rolling preformed webs having regions of non-uniform basis weight, density, or composition.

The present invention includes means by which the sheet can be independently densified in the high and low basis weight zones. This allows the apparent sheet caliper to be designed to be uniform under the pressures of layering, resulting in a layered bundle that is not only uniformly wound or stacked, but also that obviates problems with web wrinkling and binding. Without the ability to independently control the density of different regions of the web during processing, forming the bundle would be very difficult, and this particular preformed core product would not be practical.

Referring again to FIG. 6, the wide web may be differentially densified and or embossed, such as by the differential calendering described above, while the web is still on the forming wire, position 39, or after the web has been removed from the forming wire, position 58. The differential densification may also occur before or after the wide web is slit into the plurality of web segments.

The result of the invention is the ability to form a roll of a multitude of non-woven webs, each having varying material properties in the cross-machine direction, and each densified to a uniform caliper and/or embossed to a uniform apparent caliper. Each of the webs is readily usable by an absorbent article manufacture as an absorbent core within a unitary diaper, feminine hygiene product, or other absorbent article. Each unitary core is formed by drawing a length of web from the roll and repeatedly slicing the web across its width into individual unitary cores.

Personal care absorbent products typically consist of a liquid permeable bodyside liner, a liquid impervious backsheet, and an absorbent core, disposed between these layers. The liner and backsheet typically extend beyond the perimeter of the absorbent core and are bonded together so as to form an effective container for the absorbent material. The process of producing a usable product from the preformed core is known as conversion, and converting machines and systems are known in the art.

The preformed core of the invention may be utilized within the converting system such that the material gradients of the core are in either the longitudinal or transverse direction with respect to the converting operation. As such, the preformed core of the invention may be viewed as having material gradients in either the longitudinal or the transverse direction.

### Description of Tests

Density and Basis Weight:

To measure basis weight, samples of appropriate size for the particular core design being measured are cut from the core using an Atom SE 20 C hydraulic press from Associated Pacific Co. and an appropriately shaped die cutter blade. These samples are then weighed on a laboratory balance to the nearest 0.001 g. Then the apparent caliper is measured using an Emveco™ 200A electronic microgauge. Density and basis weight are calculated directly from these measurements.

Density of embossed regions of a web are calculated based upon the total amount of web material within a total volume defined by the embossed material. Thus, the volume of the embossed web includes the open space between and within the ridges of the web. Therefore, the act of embossing a web region essentially decreases the density of that region by increasing the volume occupied by that portion of the web without actually decreasing the density of the actual web material. Density of the overall region is what is referred to below.

### EXAMPLES

### Example 1

A core material was formed on a conventional airlaid machine from a mixture of cellulosic fiber and superabsorbent granules at a basis weight of 450 gsm containing 55% SAP. Portions of the laydown from various combinations of forming heads was blocked by dragging 60 mm wide strips of polypropylene tape on the surface of the web below each forming head. The strips prevent the laydown from occurring in the web under the strips wherever they were located. Three samples were produced, each with a different basis weight aspect ratio due to a different combination of heads being blocked. For each sample, a web was generated with a series of alternating high basis weight zones 75 mm wide and low basis weight zones 60 mm wide. The web was slit into a core that was 175 mm wide with a 75 mm wide high basis weight zone centered between adjacent slits and low basis weight zones along the lateral edges providing areas in each zone large enough to take measurements. The web was initially densified using a uniformly heated calender roll of uniform diameter. Differences in caliper and density were the result of gradients in the sheet formation interacting with this uniform calender hardware.

These sample rolls were then tested to determine the aspect ratio of each using the test procedures described above. Samples were cut from the high basis weight zones and low basis weight zones of each core and tested. The aspect ratios for each sample are listed below:

Sample 1

Basis weight aspect ratio: 1.3

Density aspect ratio: 1.5

Caliper aspect ratio: 1.1

Sample 2

Basis weight aspect ratio: 1.8

Density aspect ratio: 2.4

Caliper aspect ratio: 1.3

Sample 3

Basis weight aspect ratio: 2.1

Density aspect ratio: 2.4

Caliper aspect ratio: 1.2

Then, the materials were differentially densified using a laboratory-scale driven calender roll that was wrapped with tape to a caliper of 0.264 mm in a pair of zones located so that they would press the low basis weight zones of the sample. The high basis weight zone of the sample was aligned to a section of the calender that had no tape wrapped around it. The air pressure on the air loaded upper roll of the calender was adjusted using an iterative process until the samples attained a uniform caliper after one pass through the calender. Sample 1 used a pressure of 100 psi, Sample 2 required a pressure of 75 psi, and sample 2 required a pressure of 50 psi.

Sample 1 after differential densification:

Basis weight aspect ratio: 1.3

Density aspect ratio: 1.3

Caliper aspect ratio 1.0

Sample 2 after differential densification:

Basis weight aspect ratio: 1.8

Density aspect ratio: 1.8

Caliper aspect ratio: 1.0

Sample 3 after differential densification:

Basis weight aspect ratio: 2.1

Density aspect ratio: 2.1

Caliper aspect ratio: 1.0

By differentially densifying the sheet, the apparent caliper was made to be uniform where it would not have been without this differential densification process.

Measurements were taken of the dimensional length of the gradient between the blocked and unblocked regions using a light table. The change in basis weight was very clearly delineated by the change in light transmission through the material. The width of the gradient for each sample was measured at 6 mm. This indicates that the maximum gradients achieved for each property in this example to be as follows:

Basis Weight Aspect Ratio Gradient: 0.35/mm

Density Aspect Ratio Gradient: 0.35/mm

### Example 2

The cores from sample 2 in Example 1 were placed in the same calender with the same configuration of wrapped tape as in Example 1. This time, the air pressure was increased to 150 psi in order to cause the density of the low and high basis weight zones to be much more similar as the low basis weight regions were compressed to a caliper less than the uniform web caliper, that of the high basis weight regions. The aspect ratios were as follows:

Sample 2 after differential densification at a higher pressure:

Basis weight aspect ratio: 1.8

Density aspect ratio: 1.3

Caliper aspect ratio: 1.4

The densities of the low and high basis weight zones were much more similar than in Example 1. Then a Fiskars hand paper crimping tool, found typically in craft stores, was used to crimp the low basis weight region of the sample. The crimping increased the apparent caliper resulting in a uniform apparent caliper across the entire sheet. The aspect ratios are given below:

Sample 2 after crimping:

Basis weight aspect ratio: 1.8

Density aspect ratio: 1.8

Apparent caliper aspect ratio: 1.0

### Example 3

Sample 2 from Example 1 was differentially calendered using a taping pattern of closely spaced parallel stripes. The portions of the calender roll that pressed the low basis weight zones were taped in a pattern of 12 mm wide axial stripes. The pattern was designed to result in 12 mm wide parallel strips in the low basis weight portion of the core which had the same caliper as the high basis weight portion separated by 12 mm strips that had a thinner caliper and higher density. The aspect ratios that resulted are as follows:

Sample 2 after striped differential calendering:

Basis weight aspect ratio: 1.8

Density aspect ratio: 1.8

Apparent caliper aspect ratio: 1.0

The closely spaced thick portions of the low basis weight zone caused the sheet to have an apparent caliper that was uniform even though other portions of the low basis weight zone were actually at a lesser caliper.

### Example 4

A core was made by forming a web of cellulosic fiber mixed with SAP granules using a conventional airlaid process. The web was formed at 650 gsm with 55% SAP. A 150 mm wide portion of the web was made to a lower basis weight at a location 205 mm away from a second low basis weight zone which was 25 mm wide. This reduction in basis weight was achieved by dragging strips of silicone coated paper of varying widths on the surface of the web below certain forming heads. The laydown of material from the affected heads did not happen in places covered by the strips resulting in reduced basis weights in these zones. This web was slit to a width of 390 mm with one slit aligning with the edge of the 150 mm wide low basis weight portion so the web contained most of both low basis weight regions. This made a preformed material suitable for a transversely converted training pant or diaper core. The resulting core had a density gradient in the longitudinal direction rather than the transverse direction.

Process conditions were chosen so that the basis weight and the density aspect ratios were identical resulting in a caliper aspect ratio of 1.0. As a result of these process conditions, a cylindrical roll was produced which did not display wrinkling, poor roll formation, or tracking problems. The roll was successfully slit to produce the necessary slit widths to execute this type of design.

Basis weight aspect ratio: 1.2

Density aspect ratio: 1.2

Caliper aspect ratio: 1.0

This design provides a core that has a target area with roughly 9% higher basis weight in the targeted zone than a core of the same overall dimensions containing the same amounts of material only with a uniform basis weight. Those skilled in the art know that this type of core design can yield a product with improved leakage performance using less total absorbent material.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A method of forming a non-woven structure having varying material properties from a continuous non-woven web comprising
creating a continuous non-woven web having material properties which vary about the web, resulting in a web having regions of differential caliper;
differentially densifying the web; and
layering the web to form a bundle.

2. The method of claim 1, wherein the web is differentially densified by the differential application of at least one of heat and pressure to the web such that the resulting caliper of the web is substantially uniform.

3. The method of claim 2, wherein the differential application of at least one of heat and pressure is applied by a calender roll having differential diameters along its length.

4. The method of claim 2, wherein the differential application of at least one of heat and pressure is applied by at least two sets of calender rolls wherein one of the rolls in the second set of calenders is segmented into separated rings having differing diameters.

5. The method of claim 2, wherein the differential application of at least one of heat and pressure is applied by a moving belt.

6. The method of claim 2, wherein the web has a basis weight aspect ratio of between 1.0 and 15.

7. The method of claim 6, wherein the web has a basis weight aspect ratio of between 1.0 and 2.1.

8. The method of claim 2, wherein the web has a basis weight aspect ratio gradient of between 0.01 to 0.5/mm.

9. The method of claim 8, wherein the web has a basis weight aspect ratio gradient of between 0.01 and 0.35/mm.

10. The method of claim 2, wherein the web has a basis weight gradient of between about 1 and about 50 gsm/mm.

11. The method of claim 10, wherein the web has a basis weight gradient of between 1 and 44 gsm/mm.

12. The method of claim 2, wherein the web has a weight aspect ratio and density aspect ratio that are within 20% of one another after the web is differentially densified, and prior to the web being layered.

13. The method of claim 2, wherein the web has a weight aspect ratio and density aspect ratio that are within 20% of one another after the web is differentially densified, and subsequent to the web being layered.

14. The method of claim 1, wherein the material properties which vary along the web are selected from basis weight, density, and composition.

15. The method of claim 1, further comprising the step of differentially embossing the web such that the resulting web has a substantially uniform apparent caliper.

16. The method of claim 15, wherein the step of differentially embossing the web occurs in conjunction with the differential densification step.

17. The method of claim 15, wherein the step of differentially embossing the web occurs subsequent to the differential densification step.

18. The method of claim 15, wherein the embossing step is applied to one surface of the web.

19. The method of claim 15, wherein the embossing step is applied to both surfaces of the web.

20. The method of claim 15, wherein a textured calender roll is used to differentially emboss the web.

21. The method of claim 1, further comprising slitting the web into a plurality of web segments such that the material properties of each web segment vary from a first region of the segment to a second region of the segment, wherein each web segment has a uniform caliper.

22. The method of claim 15, further comprising slitting the web into a plurality of web segments such that the material properties of each web segment vary from a first region of the segment to a second region of the segment, wherein each web segment has a uniform apparent caliper.

23. The method of claim 1, wherein the web is wound into a roll which is substantially cylindrical.

24. The method of claim 1, further comprising the step of unwinding the wound roll and cross-cutting the web into unitary preformed cores.

25. The method of claim 24, further comprising the step of introducing the preformed core to a converting apparatus capable of producing absorbent articles incorporating the preformed core such that the material property gradient of the core is in the longitudinal direction with respect to the finished article.

26. The method of claim 24, further comprising the step of introducing the preformed core to a converting apparatus capable of producing absorbent articles incorporating the preformed core such that the material property gradient of the core is in the transverse direction with respect to the finished article.

27. The method of claim 21, wherein the step of layering comprises an operation selected from the group comprising winding upon a roll, winding upon a spool, and festooning.

28. A preformed absorbent core comprising a body formed of non-woven fibers having a uniform caliper and varying material property of at least one of basis weight, density, and composition, wherein at least a portion of the non-woven body is embossed.

29. The preformed absorbent core of claim 28, wherein the basis weight aspect ratio and density aspect ratio are within 20% of one another.

30. The preformed absorbent core of claim 28, wherein the composition and basis weight of the web is uniform while the density has a gradient in the cross direction.

31. An absorbent article comprising the preformed absorbent core of claim 28 positioned within an absorbent article such that the at least one material property varies in the longitudinal direction with respect to the finished article.

32. An absorbent article comprising the preformed absorbent core of claim 28 positioned within an absorbent article such that the at least one material property varies in the transverse direction with respect to the finished article.
